(19) United States
(12) Patent Application Publication
(10) Pub. No.: US 2005/0054923 A1
(43) Pub. Date: Mar. 10, 2005

(54) Method for remote consultation via mobile communication apparatus and system thereof

(76) Inventor: William Pan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/801,709

(22) Filed: Mar. 17, 2004

(30) Foreign Application Priority Data
Sep. 9, 2003 (TW) 92124936

(51) Int. Cl. A61B 8/00

(52) U.S. Cl. 600/437

(57) ABSTRACT

A system for remote consultation via a remote mobile communication apparatus has a unit of medical equipment, a medical server and a remote mobile communication apparatus. A method for remote consultation employs the medical equipment to produce the first medical report and store it into the medical server, transmits the first medical report with the medical server to the remote mobile communication apparatus via a wireless communication network, browses the first medical report, increases, modifies or varies the medical image or medical text of the first medical report to form the second medical report, and uses the mobile communication apparatus to send the second medical report back to the medical server via the wireless communication network. The remote consultation can be reached.

### FIELD OF THE INVENTION

The present invention is directed to a method for remote consultation via a mobile communication apparatus and a system thereof, and more particularly, to a method and system that is used to transmit medical reports from a hospital to a remote mobile communication apparatus for remote consultation.

### BACKGROUND OF THE INVENTION

With the progress in computer science, computer technology has been applied to the medical equipments to improve medical techniques. For instance, the 3-D image techniques have been applied in the ultrasound instrument to obtain the inner image of the human body and the face or image of a baby. Further, the computer technology is also applied to the tomography scanner or endoscopic camera to digitalize the images obtained for storage or further application. Employing the computer to compose the images can quickly reveal the cause of disease and make the success rate of a medical operation increase considerably.

However, in some medical processes or before an important operation, generally, several experienced doctors will need to consult with each other to decide the medical method or operation method. The traditional way is to invite local, national or international, well-known doctors to consult together at a specific time, but this consumes time very much. Besides, for some urgent medical cases, this way usually impedes optimal medical timing.

Further, the mobile communication apparatus, such as cellular phone, is quite popular at present. With the progress of communication techniques and the increase of the transmission bandwidth, some novel cellular phones can be used to send multimedia messages and can even download Java programs (mobile Java program) to increase their functions. The multimedia messages include, for example, various color pictures, animations and sounds.

Since the Java programs can be executed on different platforms, have compact machine codes suited for network transmission and have built-in security checking functions, there is a big communication company proposing Java 2 micro edition (J2ME) mobile information device profile (MIDP) as a standard mobile phone platform for developing Java programs. The software provider can use this platform to develop various application services for user to download.

Hence, by using the multimedia message service (MMS) and the mobile Java programs, the medical images mentioned above can be sent to the cellular phones of the doctors as multimedia messages or sent to a medical apparatus via the cellular phone for emergent consultation. Further, the doctors can also send back a medical report by the cellular phone. The traditional drawbacks can be removed.

### SUMMARY OF THE INVENTION

An objective of the present invention is to provide a method and system for remote consultation via a mobile communication apparatus to solve the conventional problem that doctors can't remotely perform an emergency medical consultation.

For reaching the objective above, the present invention mainly provides a method for remote consultation via a remote mobile communication apparatus. A unit of medical equipment is employed to produce the first medical report and store it in a medical server. The medical server transmits the first medical report to the remote mobile communication apparatus via a wireless communication network and displays it on a screen. The first medical report is browsed via the mobile communication apparatus. After consultation, the mobile communication apparatus is employed to increase, modify or vary the medical image or medical text of the first medical report to form the second medical report. The mobile communication apparatus is then employed to send the second medical report back to the medical server via the wireless communication network. The remote consultation can thus be conducted.

Another feature of the present invention is to provide the method for remote consultation via a remote mobile communication apparatus mentioned above, in which the mobile communication apparatus further connects with a remote medical apparatus. After employing the medical server to transmit the first medical report to the remote mobile communication apparatus, the first medical report is sent to the remote medical apparatus via the mobile communication apparatus. The first medical report is browsed via the remote medical apparatus. After consultation, the remote medical apparatus is employed to increase, modify or vary the medical image or medical text of the first medical report to form the second medical report. The second medical report is sent to the mobile communication apparatus, and the mobile communication apparatus sends the second medical report back to the medical server via the wireless communication network. The remote consultation can thus be conducted.

Still another feature of the present invention is to provide a system for remote consultation via a remote mobile communication apparatus, and comprises at least a medical equipment, a medical server, and a remote mobile communication apparatus. The medical equipment is used to produce a first medical report, the medical server is used for storing the first medical report and the remote mobile communication apparatus is able to connect with the medical server via a wireless communication network for accessing the first medical report for remote consultation and for sending a second medical report back after the second medical report is produced. The remote consultation can thus be conducted.

Still another feature of the present invention is to provide the system for remote consultation via the remote mobile communication apparatus mentioned above, which further includes a remote medical apparatus able to connect with the mobile communication apparatus for accessing the first medical report, producing the second medical report and sending the second medical report back to the mobile communication apparatus. Thereby, it can resolve the problem that the mobile communication apparatus is not convenient to operate for consultation.

Numerous additional features, benefits and details of the present invention are described in the detailed description, which follows.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 is a schematic diagram of the first embodiment in accord with the present invention;

FIG. 2 is schematic diagram of an inner structure of the medical server in accordance with the present invention;

FIG. 3 is a schematic diagram of the second embodiment in accord with the present invention;

FIG. 4 is a flowchart of the first embodiment of the method for remote consultation in accordance with the present invention; and

FIG. 5 is a flowchart of the second embodiment of the method for remote consultation in accordance with the present invention.

### DETAILED DESCRIPTION

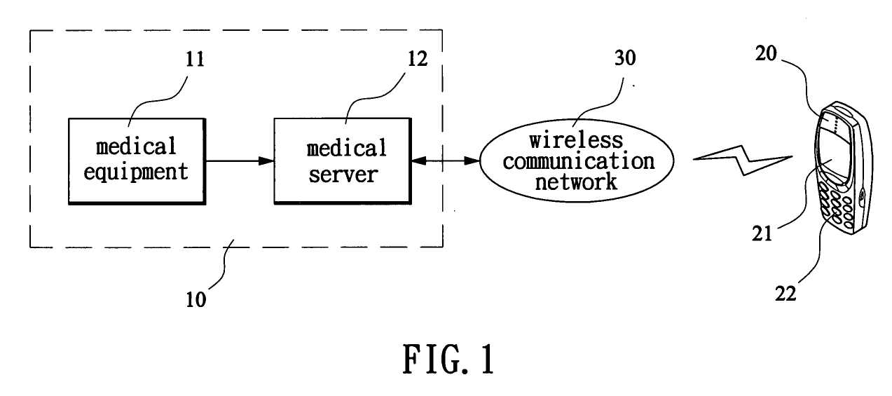

Reference is made to FIG. 1, which is a schematic diagram of the first embodiment in accord with the present invention. The system of the present invention includes a hospital end 10 and a remote end. The hospital end 10 has medical equipment 11 and a medical server 12. The remote end can employ a remote mobile communication apparatus 20 to receive the first medical report from the hospital end 10 for consultation. Then, the result of the consultation, i.e. the second medical report, can also be sent back to the hospital end 10.

In the present invention, the remote mobile communication apparatus 20 is a portable apparatus, such as a mobile phone or a personal digital assistant (PDA). The present invention takes a novel mobile phone for an example. The novel mobile phone has a larger screen 21. It can display an image message provided via the multimedia message service (MMS) or execute a Java program to download an image message. The present invention mainly uses the image displaying function of the mobile phone to show the first medical report on the screen 21 for remote consultation.

The medical equipment includes a medical image instrument and a medical report generator (not shown). The medical image instrument, such as, for example, a angiographer, ultrasound detector, endoscopic camera, intraoral camera or tomography scanner, can be used to photograph an inner image of a human body to produce at least a medical image. On the other hand, the medical report generator is used to combine the medical images with medical text to generate a medical report, and is, for example, a computer.

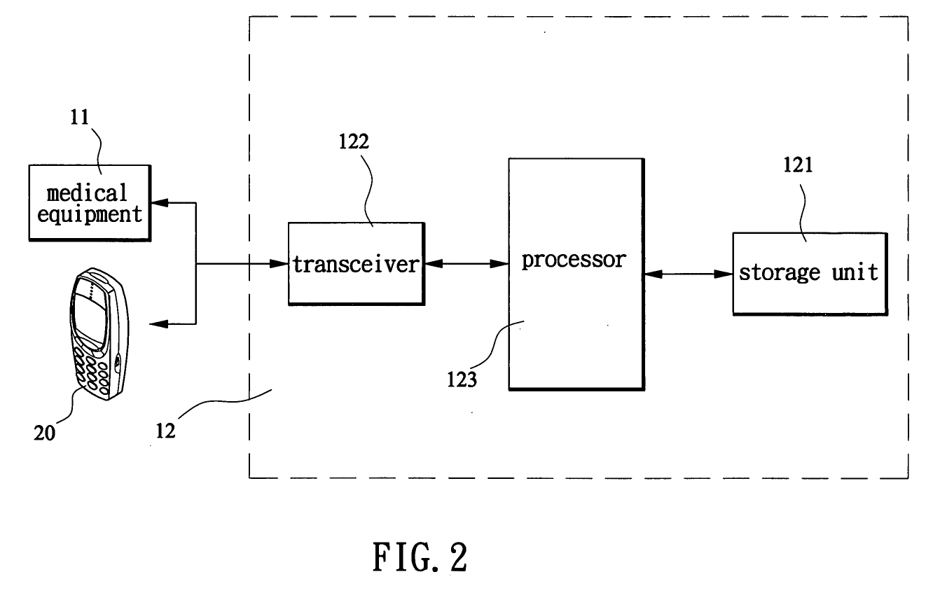

Reference is made to FIG. 1 together with FIG. 2, which is schematic diagram of an inner structure of the medical server in accordance with the present invention. The medical server 12 is connected with the medical equipment 11 for storing the first medical report generated by the medical equipment 11 or the second medical report sent from the remote end. The medical server 12 primarily includes a storage unit 121, a transceiver 122 and a processor 123. The storage unit can be used to store the medical reports. The transceiver 122 can be used to send the first medical report to the remote mobile communication apparatus 20 or receive the second medical report from the remote mobile communication apparatus 20 via the wireless communication network 30. The processor 123 is connected with the storage unit 121 and transceiver 122 for controlling the accessing, searching, storing or transmitting of the first or second medical report.

A user can use the remote mobile communication apparatus 20 to connect with the medical server 12 to access the first medical report for remote consultation and then send back the second medical report via the wireless communication network 30. The wireless communication network 30 can be a GSM system, GPRS system or 3G system. The remote mobile communication apparatus 20 can access the first medical report or send back the second medical report via employing the multimedia message service (MMS) or execute a Java program to download the first medical report or send back the second medical report.

The remote mobile communication apparatus 20 can include the screen 21 for displaying the first medical report and an input unit 22 for browsing the first medical report or modifying the medical image or text of the first medical report to form the second medical report. The input unit 22 of the remote mobile communication apparatus 20 can include a magnifying, minifying, page-up or page-down key. Hence, a user can use the magnifying, minifying, page-up or page-down key to browse the medical images. Additionally, the user can also use text input keys or handwriting input device to input, increase or modify the medical text to form the second medical report.

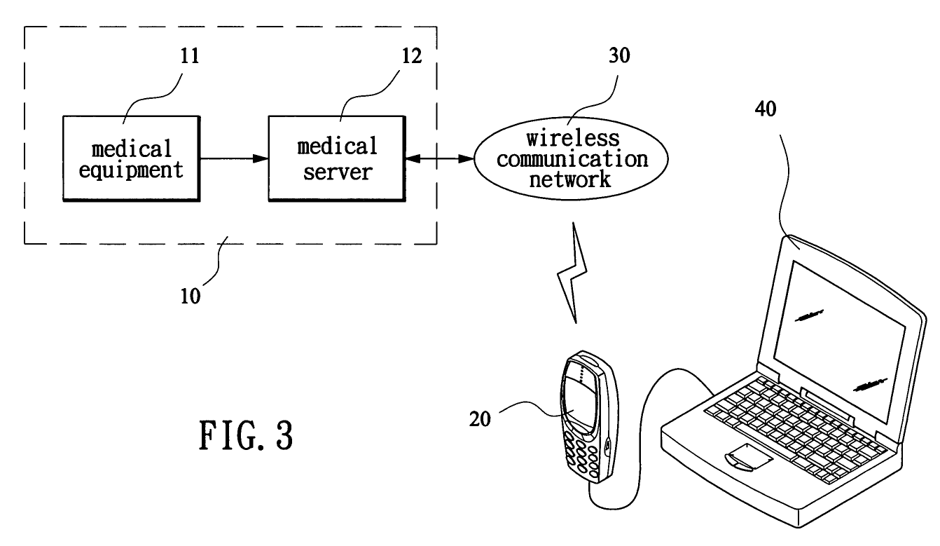

In the first embodiment of the present invention, although a doctor can use the remote mobile communication apparatus 20, such as a mobile phone or PDA, to compose the second medical report, this is still very inconvenient. Hence, the present invention connects the remote mobile communication apparatus 20 with a remote medical apparatus 40, as shown in FIG. 3, which is a schematic diagram of the second embodiment in accord with the present invention. The remote medical apparatus 40 can connect with the remote mobile communication apparatus 20 in a wireless or wired manner. Hence, a user can use it to access the first medical report in the remote mobile communication apparatus 20, generate the second medical report and send the second medical report back to the remote mobile communication apparatus 20, conveniently.

The remote medical apparatus 40 can be a computer, which mainly includes a medical report displaying device and a medical report generating device (not shown). The medical report displaying device can be used to browse the first medical report. Hence, it also includes the magnifying, minifying, page-up or page-down key. On the other hand, the medical report generating device can be used to increase, modify or vary the medical image or medical text of the first medical report to form the second medical report.

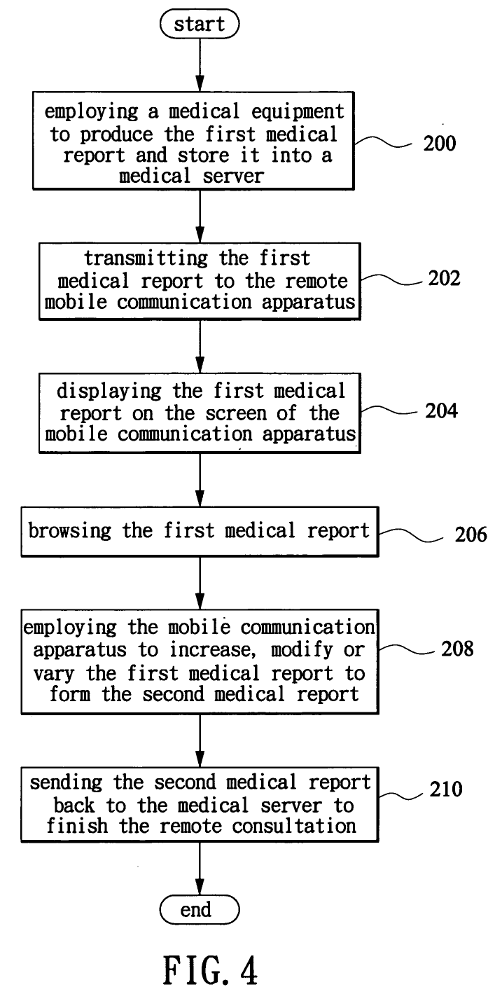

Reference is made to FIG. 4, which is a flowchart of the first embodiment of the method for remote consultation in accordance with the present invention. In the present invention, the first embodiment of the method for remote consultation includes following steps:

Step 200: first, employing the medical equipment 11 to produce the first medical report and store it into the medical server 12;

Step 202: employing the medical server 12 to transmit the first medical report to the remote mobile communication apparatus 20 via the wireless communication network 30;

Step 204: displaying the first medical report on the screen 21 of the mobile communication apparatus 20;

Step 206: browsing the first medical report via the mobile communication apparatus 20 for remote consultation;

Step 208: after consultation, employing the input unit 22 of the mobile communication apparatus 20 to increase, modify or vary the medical image or medical text of the first medical report to form the second medical report; and

Step 210: employing the mobile communication apparatus 20 to send the second medical report back to the medical server 12 via the wireless communication network 30 to finish the remote consultation.

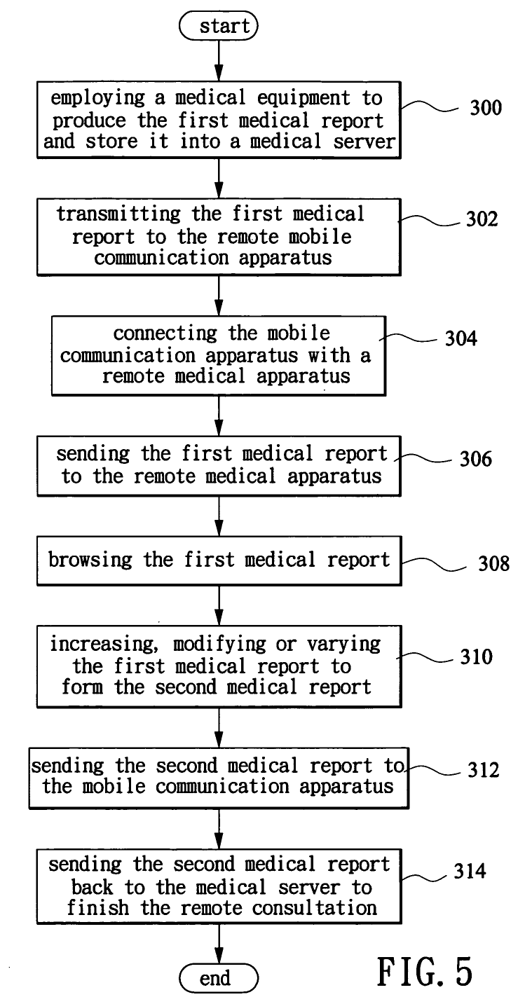

Reference is made to FIG. 5, which is a flowchart of the second embodiment of the method for remote consultation in accordance with the present invention. In the present invention, the second embodiment of the method for remote consultation includes following steps:

Step 300: first, employing the medical equipment 11 to produce the first medical report and store it into the medical server 12;

Step 302: employing the medical server 12 to transmit the first medical report to the remote mobile communication apparatus 20 via the wireless communication network 30;

Step 304: connecting the mobile communication apparatus 20 with the remote medical apparatus 40;

Step 306: sending the first medical report to the remote medical apparatus 40 via the mobile communication apparatus 20;

Step 308: browsing the first medical report via the remote medical apparatus 40 for remote consultation;

Step 310: after consultation, employing the remote medical apparatus 40 to increase, modify or vary the medical image or medical text of the first medical report to form the second medical report;

Step 312: sending the second medical report to the mobile communication apparatus 20; and

Step 314: employing the mobile communication apparatus 20 to send the second medical report back to the medical server 12 via the wireless communication network 30 to finish the remote consultation.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are embraced within the scope of the invention as defined in the appended claims.

1. A method for remote consultation, comprising:
employing a medical server to transmit a first medical report to a remote mobile communication apparatus via a wireless communication network;
browsing the first medical report so as to produce a second medical report; and
sending the second medical report back to the medical server.

2. The method as claimed in the claim 1, wherein the mobile communication apparatus is a portable apparatus.

3. The method as claimed in the claim 1, wherein the step of employing the medical server further comprises:
employing at least a unit of medical equipment to produce the first medical report; and
storing the first medical report into the medical server.

4. The method as claimed in the claim 3, wherein the unit of medical equipment has a medical image instrument used to photograph an inner image of a human body to produce at least a medical image.

5. The method as claimed in the claim 3, wherein the unit of medical equipment has a medical report generator used to combine medical images with a medical text to generate the first medical report.

6. The method as claimed in the claim 5, wherein the medical report generator is a computer.

7. The method as claimed in the claim 1, wherein the step of employing the medical server to transmit the first medical report to the remote mobile communication apparatus uses a multimedia message service (MMS) to transmit the first medical report.

8. The method as claimed in the claim 1, wherein the step of employing the medical server to transmit the first medical report to the remote mobile communication apparatus executes a Java program to download the first medical report from the medical server.

9. The method as claimed in the claim 1, wherein the first medical report comprises at least a medical image and a medical text.

10. The method as claimed in the claim 1, wherein the step of employing the medical server to transmit the first medical report to the remote mobile communication apparatus further comprises:
displaying the first medical report on a screen of the mobile communication apparatus.

11. The method as claimed in the claim 1, wherein the step of browsing the first medical report is performed by using an input unit of the mobile communication apparatus.

12. The method as claimed in the claim 11, wherein the step of browsing the first medical report is performed by using a magnifying key, a minifying key, a page-up key or a page-down key of the input unit of the mobile communication apparatus.

13. The method as claimed in the claim 1, wherein the step of browsing the first medical report so as to produce the second medical report is performed by using an input unit of the mobile communication apparatus to increase, modify or vary a medical image or a medical text of the first medical report to form the second medical report.

14. The method as claimed in the claim 13, wherein the input unit is a text input key or a handwriting input device.

15. The method as claimed in the claim 1, wherein the step of browsing the first medical report comprises:
connecting the mobile communication apparatus with a remote medical apparatus;
sending the first medical report to the remote medical apparatus;
consulting by using the first medical report and producing the second medical report by using the remote medical apparatus; and
sending the second medical report to the mobile communication apparatus.

16. The method as claimed in the claim 15, wherein the mobile communication apparatus is connected with the remote medical apparatus in a wired or a wireless manner.

17. The method as claimed in the claim 15, wherein the remote medical apparatus has a medical report displaying device used for a user to browse the medical reports.

18. The method as claimed in the claim 15, wherein the remote medical apparatus has a medical report generating device used to increase, modify or vary a medical image or a medical text of the first medical report to form the second medical report.

19. The method as claimed in the claim 15, wherein the remote medical apparatus is a computer.

20. A system for remote consultation, comprising:
at least a unit of medical equipment used to produce a first medical report;
a medical server connected with the medical equipment for storing the first medical report; and
a remote mobile communication apparatus connected with the medical server via a wireless communication network for accessing the first medical report for remote consultation and sending a second medical report back after the second medical report is produced.

21. The system as claimed in the claim 20, wherein the unit of medical equipment has a medical image instrument used to photograph an inner image of a human body to produce at least a medical image.

22. The system as claimed in the claim 21, wherein the medical image instrument is an angiographer, an ultrasound detector, an endoscopic camera, an intraoral camera or a tomography scanner.

23. The system as claimed in the claim 20, wherein the unit of medical equipment has a medical report generator used to combine medical images with a medical text to generate the first medical report.

24. The system as claimed in the claim 23, wherein the medical report generator is a computer.

25. The system as claimed in the claim 20, wherein the first medical report comprises at least a medical image and a medical text.

26. The system as claimed in the claim 20, wherein the medical server comprises:
a storage unit used to store the first medical report or the second medical report;
a transceiver used to transmit the first medical report to the remote mobile communication apparatus via the wireless communication network or receive the second medical report from the remote mobile communication apparatus; and
a processor connected with the storage unit and the transceiver for accessing, searching, storing, or transmitting the first medical report or the second medical report.

27. The system as claimed in the claim 20, wherein the mobile communication apparatus is a portable apparatus.

28. The system as claimed in the claim 20, wherein the remote mobile communication apparatus uses a multimedia message service (MMS) to access the first medical report or send the second medical report back.

29. The system as claimed in the claim 20, wherein the remote mobile communication apparatus executes a Java program to download the first medical report from the medical server or send the second medical report back.

30. The system as claimed in the claim 20, wherein the remote mobile communication apparatus comprises:
a screen for displaying the first medical report; and
an input unit for browsing the first medical report so as to increase, modify or vary a medical image or a medical text of the first medical report to form the second medical report.

31. The system as claimed in the claim 30, wherein the input unit has a magnifying key, a minifying key, a page-up key or a page-down key.

32. The system as claimed in the claim 30, wherein the input unit is a text input key or a handwriting input device.

33. The system as claimed in the claim 20, further comprising:
a remote medical apparatus connected with the remote mobile communication apparatus for accessing the first medical report, producing the second medical report and sending the second medical report back to the remote mobile communication apparatus.

34. The system as claimed in the claim 20, wherein the remote medical apparatus is connected with the mobile communication apparatus in a wired or a wireless manner.

35. The system as claimed in the claim 34, wherein the remote medical apparatus comprises:
a medical report displaying device used for a user to browse the medical reports; and
a medical report generating device used to increase, modify or vary a medical image or a medical text of the first medical report to form the second medical report.

36. The system as claimed in the claim 35, wherein the medical report displaying device has a magnifying key, a minifying key, a page-up key or a page-down key.

37. The system as claimed in the claim 34, wherein the remote medical apparatus is a computer.
